(19) United States
(12) Patent Application Publication
(10) Pub. No.: US 2026/0224406 A1
(43) Pub. Date: Aug. 6, 2026

(54) MEDICAL DRESSINGS WITH THERMOCHROMIC INDICATORS

(71) Applicant: Solventum Intellectual Properties Company, Maplewood, MN (US)

(72) Inventors: Ying ZHANG, Minneapolis, MN (US); Done DEMIRGOZ, Minneapolis, MN (US); Zane G. JOHNSON, Minneapolis, MN (US); Kristopher E. HUNTER, Minneapolis, MN (US); Jason W. BJORK, Minneapolis, MN (US); David R. HOLM, Minneapolis, MN (US)

(73) Assignee: Solventum Intellectual Properties Company, Maplewood, MN (US)

(21) Appl. No.: 19/157,245

(22) PCT Filed: Feb. 2, 2024
(86) PCT No.: PCT/IB2024/050982

§ 371 Date: Aug. 15, 2025

Related U.S. Application Data
(60) Provisional application No. 63/486,936, filed on Feb. 24, 2023.

(51) Int. Cl. A61F 13/00; A61F 13/02; A61L 26/00

(52) U.S. Cl. CPC A61F 13/00059; A61F 13/00055; A61F 13/00063; A61F 13/0263; A61L 26/0066; A61L 26/008; A61F 2013/00412; A61F 2013/00676; A61F 2013/00953; A61L 2300/404

(57) ABSTRACT

A medical dressing. The medical dressing includes a backing layer comprising a first major surface and a second major surface opposite the first major surface; an adhesive on the second major surface of the backing layer; a release liner; and a thermochromic indicator having a color-change sensitivity at a temperature in a range between about 20.0° C. to about 45 C.

### FIELD

The present disclosure relates to a medical dressing with a thermochromic indicator.

### BACKGROUND

Intravenous (IV) infusion as a delivery means of a biological fluid to a patient is a common clinical procedure in medical settings. In the United States, approximately 80% of hospitalized patients receive IV therapy. In general, a needle or cannula is inserted into a peripheral vein and connected to an IV bag containing a biological fluid by means of a flexible connector tube. The biological fluid can be mere saline solution and/or contain any of the numerous therapeutic agents and/or nutrients administrable by IV infusion.

Transparent film dressings are widely used as protective layers over wounds because they facilitate healing in a moist environment while acting as a barrier to contaminating liquids and bacteria. The films are also used as surgical drapes because of their barrier properties. Dressings and drapes fitting the above description are available under a number of trade names such as TEGADERM™ (3M Company, St. Paul, Minn.) and OP-SITE™ (Smith & Nephew, Hull, England). The polymeric films used in those dressings and drapes are conformable, i.e., the films are extremely thin, flexible and supple. They are typically supplied with a releasable protective liner covering the adhesive coated surface of the film.

The use of a removable carrier, which does not require tearing of the film after it has been placed on the patient, avoids the problems described above. The carrier also aids in accurate placement of the dressing on a patient. There is a need for a better medical dressing and nonclinical approaches to detect infiltrations.

### SUMMARY

At times, and due to various causes, there is a need of a smart dressing to detect temperature change of mammalian body's skin or subcutaneous tissues. For example, the IV fluid infiltrates nonvascular tissue proximate to the site of injection or infusion, adversely affecting the surrounding tissue and disrupting the therapeutic schedule. Further, the infiltration or extravasation can permeate tissue distal to the site of IV infusion, such as in the vicinity of the venous drainage system of the IV infusion. The IV solution can accumulate in the skin and subcutaneous tissue, possibly damaging the skin and tissues and preventing critical medication and/or nutrients from reaching the venous system.

Thus, in one aspect, the present disclosure provides a medical dressing comprising: a backing layer comprising a first major surface and a second major surface opposite the first major surface; an adhesive on the second major surface of the backing layer; a release liner; and a thermochromic indicator having a color-change sensitivity at a temperature in a range between about 20.0° C. to about 45 C.

In another aspect, the present disclosure provides a method comprising providing the medical dressing of the present disclosure; applying the medical dressing to a mammalian body; detecting a color change that develops from an injection into the mammalian body.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF THE DRAWINGS

FIG. 1 is a side sectional view of one illustrative embodiment of a medical dressing as described herein.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

Smart dressings are needed to detect temperature change of mammalian body's skin or subcutaneous tissues. For example, various nonclinical approaches to detect infiltrations have been proposed. So far, these nonclinical methods have been largely unsuccessful. Resistance to flow and/or increased pressure within the IV connector tube is monitored by a detector emitting an alarm when flow resistance or pressure increases above a threshold value. The medical dressing of the present disclosure, in which a thermochromic indicator is incorporated into the medical dressing, can provide a new apparatus system useful for indicating temperature change of a mammalian body's skin or subcutaneous tissues to detect the occurrence of an infiltration or extravasation during the IV administration of a biological fluid to a mammalian body, such as a patient body, or phlebitis or to tell users how to apply medical dressing correctly or detect edge lift of the medical dressing indicating.

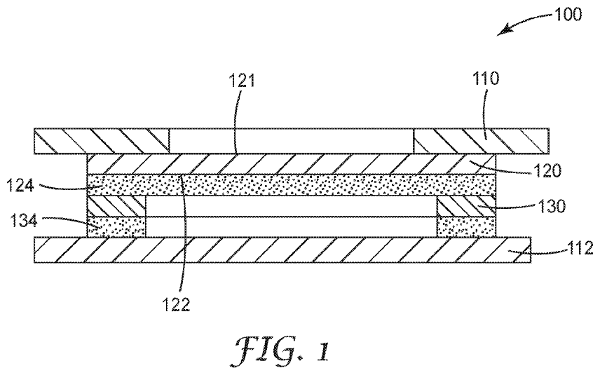

FIG. 1 depicts one illustrative embodiment of a medical dressing 100 as described herein. In one or more embodiments, the medical dressing 100 may be described as including a backing layer 120 having a first major surface 121 and a second major surface 122 opposite the first major surface 121. The medical dressing 100 also includes adhesive 124 located on the second major surface of the backing layer 120, and a support material 130 secured to the second major surface 122 of the backing layer 120. In one or more embodiments, the medical dressing 100 may include adhesive 134 on the surface of the support material 130 that faces away from the backing layer 120 such that both the backing layer 120 and the support material 130 may be adhered to the skin of a patient. In one or more embodiments, the medical dressing 100 may also include a release liner 112 and a carrier 110 releasably attached to the first major surface of the backing layer. In one or more embodiments, the medical dressing 100 may also include a hydrogel island pad (not shown) proximate the second major surface of the backing. In some embodiments, the hydrogel island pad can be secured to the second major surface 122 of the backing layer 120 by the adhesive 124 located on the second major surface of the backing layer 120. In some embodiments, the hydrogel island pad can be secured to the second major surface 122 of the backing layer 120 by the adhesive 134 on the surface of the support material 130 that faces away from the backing layer 120. In one or more embodiments, the medical dressing 100 may also include a thermochromic indicator having a color-change sensitivity at a temperature in a range between about 20° C. to about 45° C. or between about 25° C. to about 35° C.

In one or more embodiments, the thermochromic indicator can be a dye, colorant, ink, or pigment. In some embodiments, the dye can be a colored substance, the color of which can be detected by the human eye and/or by colorimetric sensors. Examples of a suitable thermochromic indicator can include any dyes changing color at temperature between 20° C.-45° C., for example, blue-violet with transition temperature 22° C., green-yellow, black-yellow, red-yellow, black-colorless, black-pink, black-blue, black-green, pink-colorless, yellow-colorless, black-purple with transition temperature 25° C., black-colorless or red-colorless with transition temperature 28° C., blue-violet, red-yellow, black-pink, black-yellow, red-colorless, blue-colorless, black-colorless with transition temperature 31° C., red-colorless with transition temperature 29° C., black-colorless, black-yellow, black-green, black-pink, black-purple with transition temperature 35° C., black-colorless with transition temperature 38° C., red-colorless with transition temperature 45° C. In some embodiments, the thermochromic indicator can be a powder or dissolved or suspended in a solution. For example, leuco dyes, is a powder pigment that can be mixed into a liquid material (water, uncured adhesive, clear paint, release coating solution? etc.) before being applied to the medical dressing.

The medical dressings described herein may be made by conventional techniques (e.g., extrusion, solvent casting, calendaring, laminating, adhesive coating, and the like) which are familiar to those skilled in the art. U.S. Pat. No. 6,685,682, the disclosure of which is herein incorporated by reference, discloses some potentially useful constructions and methods for making medical dressings with backing layers and support material as described herein. In one or more embodiments, the thermochromic indicator can be incorporated in or located on any portion of the medical dressing, for example, the backing layer, the adhesive, the release liner, the support material, the hydrogel island pad, or the carrier of the medical dressing. In some embodiments, the thermochromic indicator can be transferred from the carrier to the backing layer upon removal of the carrier. In some embodiments, the thermochromic indicator can be printed on the medical dressing, for example, on the backing layer, the adhesive, the release liner, the support material, the hydrogel island pad, or the carrier of the medical dressing. In some embodiments, thermochromic indicator can partially or fully cover or form a pattern on the backing layer, the adhesive, the release liner, the support material, the hydrogel island pad, or the carrier of the medical dressing.

The thermochromic indicator can indicate a change in temperature from a temperature indicative of a normal skin to a temperature indicative of IV fluid infiltration. Therefore, the medical dressing of present disclosure can pick up a temperature change induced by cool fluids entering the tissue subcutaneously, for example, in the case of infiltration at an IV site. Transition temperature of the thermochromic indicator can be chosen to be less than surface skin temperature, for example, in a range between about 20.0° C. to about 35° C., so that when the fluids cool the tissue, the skin surface temperature drops below the transition temperature, changing the color of the thermochromic indicator. In some embodiments, the medical dressing can be a primary or secondary medical dressing such as those used for; IV site dressings (for example, IV catheter dressing), surgical dressings, general wound dressings, dressings that may or may not absorb fluid, dressings that may or may not contain gels or hydrogels, dressings used to prevent skin injury such as abrasion, dressings that may or may not contain antimicrobials, dressings that may or may not contain hemostatic agents, transparent or non-transparent dressings.

The present disclosure also provides a method to detect the occurrence of an infiltration or extravasation. The method can include providing the medical dressing of the present disclosure, applying the medical dressing to a mammalian body, and detecting a color change that develops from an injection into the mammalian body. The method may also include inserting a hypodermic needle or cannula through a skin of the mammalian body or into a blood vessel of the mammalian body that is covered by the medical dressing. The color change can be detected by the human eye and/or by colorimetric detection device, for example, colorimetric sensors or RGB-IR camera. The present disclosure provides an easy solution without the need of additional tools or apparatus other than thermochromic indicator incorporated into the medical dressing. The medical dressing of the present disclosure can allow for non-invasive constant passive temperature monitoring that helps to identify the location and sizes of infiltration or extravasations in real time using visible color changes. The existing technologies to detect infiltration such as electrical impedance, light absorption changes require separate device, while the medical dressing of the present disclosure can serve as a securement dressing providing securement of catheters and a means to detect IV related complications, infiltration and extravasation. The medical dressing of the present disclosure can also be used to detect phlebitis, thrombophlebitis, infection, patient temperature, patient temperature changes, local injury, inflammation, wound healing changes, dressing adherence, dressing edge lift, air gaps in dressings. In some embodiments, the medical dressing of the present disclosure can provide an insulating effect of the thermochromic indicator in the dressing so the thermochromic indicator will be more impacted by temperature change of the skin/tissue and less influenced by the ambient temperature or by changes in the ambient temperature.

### Backing Layers

The backing layers of one or more embodiments of medical dressings described herein may provide an impermeable barrier to the passage of liquids and at least some gases. Representative backing layers may include non-woven and woven fibrous webs, knits, films, foams, polymeric films and other familiar backing materials. In some embodiments, a transparent backing layer is desirable to allow for viewing of the underlying skin or medical device.

In one embodiment, the backing layer has high moisture vapor permeability, but generally impermeable to liquid water so that microbes and other contaminants are sealed out from the area under the backing layer. One example of a suitable material is a high moisture vapor permeable film such as described in U.S. Pat. Nos. 3,645,835 and 4,595,001, the disclosures of which are herein incorporated by reference. In high moisture vapor permeable film/adhesive composites, the composite should transmit moisture vapor at a rate equal to or greater than human skin such as, for example, at a rate of at least 200 g/m²/24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001. Perforated substrates or films or pattern coated adhesives may be used to increase the moisture vapor transmission. In one embodiment, the backing layer is an elastomeric polyurethane, polyester, or polyether block amide films. These films combine the desirable properties of resiliency, elasticity, high moisture vapor permeability, and transparency. A description of this characteristic of backing layers can be found in issued U.S. Pat. Nos. 5,088,483 and 5,160,315, the disclosures of which are hereby incorporated by reference

Commercially available examples of potentially suitable backing layers may include the thin polymeric film backings sold under the trade names TEGADERM (3M Company), OPSITE (Smith & Nephew), etc. Many other backing layers may also be used, including those commonly used in the manufacture of surgical incise drapes (e.g., incise drapes manufactured by 3M Company under the trade names STERIDRAPE and IOBAN), etc.

Because fluids may be actively removed from the sealed environments defined by the medical dressings, a relatively high moisture vapor permeable backing layer may not be required. As a result, some other potentially useful backing materials may include, e.g., metallocene polyolefins and SBS and SIS block copolymer materials could be used.

Regardless, however, it may be desirable that the backing layer be kept relatively thin to, e.g., improve conformability. For example, the backing layer may be formed of polymeric films with a thickness of 200 micrometers or less, or 100 micrometers or less, potentially 50 micrometers or less, or even 25 micrometers or less.

### Support Materials

The support materials used in one or more embodiments of medical dressings as described herein may provide strength to the backing layer. The support material therefore has more stiffness and less elasticity than the backing layer. The support material may be a coating, such as an adhesive, or may be a self-supporting substrate such as another film, woven, knitted, or nonwoven fabric. For example, U.S. Pat. No. 5,088,483 discloses a permanent adhesive as a reinforcement that could be used as the support material.

One example of nonwoven for the support material is a high strength nonwoven fabric available from E. I. Dupont de Nemours & Company of Wilmington, Del. under the trademark Sontara, including Sontara 8010, a hydroentangled polyester fabric. Other suitable nonwoven webs include a hydroentangled polyester fabric available from Veratec, a division of International Paper of Walpole, Mass. Another suitable nonwoven web is the nonwoven elastomeric web described in U.S. Pat. No. 5,230,701.

### Adhesives

Suitable adhesive for use in one or more embodiments of the medical dressings described herein include any adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including for example an antimicrobial agent.

The pressure sensitive adhesives that may be used in the medical dressings may include adhesives that are typically applied to the skin such as the acrylate copolymers described in U.S. Pat. No. RE 24,906, particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Silicone adhesive can also be used. Generally, silicone adhesives can provide suitable adhesion to skin while gently removing from skin. Suitable silicone adhesives are disclosed in PCT Publications WO2010/056541 and WO2010/056543.

The pressure sensitive adhesives may, in some embodiments, transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as pattern coating the adhesive on the backing, as described in U.S. Pat. No. 4,595,001. Other potentially suitable pressure sensitive adhesives may include blown-micro-fiber (BMF) adhesives such as, for example, those described in U.S. Pat. No. 6,994,904. The pressure sensitive adhesive used in the wound dressing may also include one or more areas in which the adhesive itself includes structures such as, e.g., the microreplicated structures described in U.S. Pat. No. 6,893,655.

Issued U.S. Pat. Nos. 3,645,835 and 4,595,001, describe methods of making such films and methods for testing their permeability. Preferably, the film/adhesive composite should transmit moisture vapor at a rate equal to or greater than human skin. Preferably, the adhesive coated film transmits moisture vapor at a rate of at least 200 g/m²/24 hrs/37 C/100-10% RH, more preferably at least 700 g/m²/24 hrs/37 C/100-10% RH, and most preferably at least 2000 g/m²/24 hrs/37 C/100-10% RH using the inverted cup method as described in U.S. Pat. No. 4,595,001.

Different portions of the medical dressings described herein may include different adhesives, such as disclosed in US 2015/0141949 titled “Medical Dressing with Multiple Adhesives.” For example, a portion may include an acrylate adhesive while another portion may include a silicone adhesive. In one embodiment, to prevent edge separation, adjacent the perimeter is acrylate adhesive, while near the central portion there is silicone adhesive. In one embodiment, to strongly secure with a device or tubing near the central portion there is acrylate adhesive, while near the perimeter in contact with skin is silicone adhesive. In some embodiments, the medical dressings described may include multilayer of adhesive. For example one layer is continuous coated silicone adhesive and another layer is pattern coated acrylate adhesive.

### Hydrogel

A hydrogel can be a cross-linked polymer gel. Hydrogels are typically very flexible. Hydrogels provide and maintain a moist environment at the contact point on skin by increasing moisture content. Generally, hydrogels can be removed without trauma to the wound. Common ingredients are for example polyvinyl alcohol, sodium polyacrylate, acrylate polymers and copolymers with hydrophilic groups. A commercially available example of a hydrogel are Flexigel Hydrogel Sheet available from Smith & Nephew, Tegaderm CHG dressing available from 3M Company, St. Paul, Minn.

Hydrocolloids are similar to hydrogels but have the ability to absorb moisture. It is understood that for purposes of this disclosure a hydrogel or a hydrocolloid may be used. Hydrocolloids typically comprise a blend of a polymer matrix, such as a rubbery elastomer like polyisobutylene, in combination with one or more water-soluble or water-swellable hydrocolloids, such as a dry powdered mixture of pectin, gelatin and carboxymethylcellulose. Upon absorption of a liquid the hydrocolloid form a gel-like substance. In some embodiments, hydrogel can be UV curable, in which photo initiator contains in the formulation and the monomers are cured upon UV light exposure.

### Optional Components

An absorbent material may also be used in conjunction with the medical dressings described herein. An absorbent material may be the same as the wound packing material (described below) or may be a separate element. The absorbent materials can be manufactured of any of a variety of materials including, but not limited to, woven or nonwoven cotton or rayon. Absorbent pad is useful for containing a number of substances, optionally including antimicrobial agents, drugs for transdermal drug delivery, chemical indicators to monitor hormones or other substances in a patient, etc.

The absorbent may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Absorbent materials may also be chosen from other synthetic and natural hydrophilic materials including polymer gels and foams.

And optional release liners may be included that covers all or a portion of the adhesives to prevent contamination of the adhesives. In one embodiment, the package that contains the adhesive dressing may serve as a release liner. Suitable release liners can be made of kraft papers, polyethylene, polypropylene, polyester or composites of any of these materials. In one embodiment, the liners are coated with release agents such as fluorochemicals or silicones. For example, U.S. Pat. No. 4,472,480, the disclosure of which is hereby incorporated by reference, describes low surface energy perfluorochemical liners. In one embodiment, the liners are papers, polyolefin films, or polyester films coated with silicone release materials.

An optional carrier may be included that covers all or a portion of the first major surface of the substrate, providing structural support if the dressing is thin and highly flexible. The carrier maybe removable from the first major surface once the adhesive dressing is placed on skin. The carrier can be constructed of a variety of materials such as fabric that are woven or kitted, nonwoven material, papers, or film. In one embodiment, the carrier is along the perimeter of the first major surface of the dressing and is removable from the first major surface, similar to the carrier used the 3M Tegaderm™ Transparent Film Dressing, available from 3M Company, St. Paul, Minn.

An optional antimicrobial agent may be included that is either separate from the adhesive dressing or may be integral with the dressing. The antimicrobial component is placed near or adjacent to the insertion site of the medical device to inhibit microbial growth in and around the insertion site. The antimicrobial component can be absorbent foam or gel, such as used in a 3M Tegaderm™ CHG I.V. Securement Dressing, available from 3M Company. The antimicrobial agent can be selected from the group consisting of parachlorometaxylenol; triclosan; chlorhexidine and salts thereof; poly hexamethylene biguanide and salts thereof; iodine; idodophors; silver oxide; silver and its salts; octenidine; Olanexidine; peroxides; antibiotics; and combinations of the foregoing.

The following embodiments are intended to be illustrative of the present disclosure and not limiting.

### EMBODIMENTS

Embodiment 1 is a medical dressing comprising: a backing layer comprising a first major surface and a second major surface opposite the first major surface; an adhesive on the second major surface of the backing layer; a release liner; and a thermochromic indicator having a color-change sensitivity at a temperature in a range between about 20.0° C. to about 45 C.
Embodiment 2 is the medical dressing of embodiment 1, wherein the thermochromic indicator is a dye, colorant, ink, or pigment.
Embodiment 3 is the medical dressing of any of embodiments 1 to 2, wherein the thermochromic indicator is a dye.
Embodiment 4 is the medical dressing of any of embodiments 1 to 3, wherein the medical dressing is an IV site dressing.
Embodiment 5 is the medical dressing of any of embodiments 1 to 4, further comprising a support material secured to the backing layer, wherein the support material is less elastic than the backing layer.
Embodiment 6 is the medical dressing of any of embodiments 1 to 5, further comprising a hydrogel island pad proximate the second major surface of the backing.
Embodiment 7 is the medical dressing of any of embodiments 1 to 6, further comprising a carrier releasably attached to the first major surface of the backing layer.
Embodiment 8 is the medical dressing of any of embodiments 1 to 7, further comprising an antimicrobial agent.
Embodiment 9 is the medical dressing of embodiment 8, wherein the antimicrobial agent is selected from the group consisting of parachlorometaxylenol; triclosan; chlorhexidine and salts thereof; poly hexamethylene biguanide and salts thereof; iodine; idodophors; silver oxide; silver and its salts; octenidine; Olanexidine; peroxides; antibiotics; and combinations of the foregoing.
Embodiment 10 is the medical dressing of any of embodiments 1 to 9, wherein the thermochromic indicator is incorporated in or located on the backing layer, the adhesive, the release liner, the support material, the hydrogel island pad, or the carrier.
Embodiment 11 is the medical dressing of embodiment 10, wherein the thermochromic indicator is transferred from the carrier to the backing layer upon removal of the carrier.
Embodiment 12 is the medical dressing of embodiment 10, wherein the thermochromic indicator is printed on the medical dressing.
Embodiment 13 is the medical dressing of any of embodiments 1 to 12, wherein the thermochromic indicator indicates a change in temperature from a temperature indicative of a normal skin to a temperature indicative of IV fluid infiltration.
Embodiment 14 is a method comprising providing the medical dressing of any of embodiments 1 to 13; applying the medical dressing to a mammalian body; detecting a color change that develops from an injection into the mammalian body.
Embodiment 15 is the method of embodiment 14, wherein the color change indicates IV fluid infiltration.
Embodiment 16 is the method of any of embodiments 14 to 15, further comprising inserting a hypodermic needle or cannula through a skin of the mammalian body or into a blood vessel of the mammalian body that is covered by the medical dressing.

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

### Example 1

Thermochromic fabric paint solutions having color transitions of blue to colorless at 31° C., black to colorless at 28° C., and green to yellow at 25° C. were obtained from Atlanta Chemical Engineering LLC (Tampa, FL). Each solution was Meyer rod (size 28) coated onto the polyurethane film side of a 10-inch-wide adhesive laminate consisting of polyurethane film, adhesive, and release liner (3M Company, St Paul, MN) and dried overnight. Each dye-coated sample was then cut into 2 inch×2 inch dressing samples. Two dressing samples of each coated dye-coated sample were made for a total of six dressing samples.

Freshly euthanized naïve young adult female mixed breed agricultural swine (Yorkshire X from Midwest Research Swine, Gibbon, MN) with minimal skin pigmentation and weighing 10-40 kg were used. To minimize complications, hair and dirt on the swine skin at the intended application sites were removed prior to the study. A warming blanket (3M Company, St Paul, MN) was used to maintain the swine temperature throughout the study. Pairs of each coated dressing were applied to the abdomen of the pig prior to euthanasia.

After the swine was euthanized, room temperature 0.9% sodium chloride injection solution (Patterson Veterinary, Loveland CO) was injected into veins (intravenously) and subcutaneously through an either 18- or 20-gauge needle (Becton-Dickinson, Franklin Lakes, NJ) under each pair of dressings. The saline solution was injected in 2 mL increments every 10 seconds up to 12 mL. The blue to colorless dressing started to change at 2-4 mL injected subcutaneously. The black to colorless dressing also changed color after 2-4 mL of saline was injected. Each dressing was initially colorless after application and the lower temperature saline caused localized changes from colorless to blue and black, respectively. The subcutaneous injection created a color change in the shape of a diffuse oval. The intravenous injection produced a narrow, linear band of color. The remaining dressing, coated with the green to yellow at 25° C. dye, did not change during the experiment. Temperature data was collected with an IR camera (Teledyne FLIR, Wilsonville, OR) and showed that the observed color changes correlated with surface temperature changes.

### Example 2

Thermochromic fabric paint solution (blue to colorless transition at 31° C., Atlanta Chemical Engineering LLC, Tampa, FL) was Meyer rod (size 8) coated onto the polyurethane film side of an 8-inch-wide adhesive laminate consisting of polyurethane film, adhesive and release liner (3M Company, St Paul, MN), then dried in a 150° F. oven for two minutes. The dye-coated sample was then cut into 5.5 inch×5.5 inch squares to provide experimental dressing samples.

Freshly euthanized naïve young adult female mixed breed agricultural swine (Yorkshire X from Midwest Research Swine, Gibbon, MN) with minimal skin pigmentation and weighing 10-40 kg were used for this study. To minimize complications, hair and dirt on the swine skin at the intended application sites were removed prior to the study. A warming blanket (3M Company, St Paul, MN) was used to maintain the swine temperature throughout the study. A 20 gauge×1¼″ INTROCAN Safety catheter (B. Braun, Bethlehem, PA) was inserted into the right abdominal area of the swine for subcutaneous injection and then the dye coated dressing sample was applied to cover the catheter. The dressing turned colorless upon application of the dressing to the skin surface. Sodium chloride injection solution (0.9%) (Patterson Veterinary, Loveland CO) was infused into the swine at a flow rate of 100 ml/hr subcutaneously using an infusion pump (Baxter, Deerfield, IL) to simulate infiltration. The dressing started to change to color blue at approximately 2-3 ml saline infusion near the tip of the catheter, forming a diffuse oval originating at the site of tip of the catheter.

### Example 3

Polyvinylpyrolidone K90 with molecular weight approximately 1,570,000 g/mol supplied by Ashland Chemical (Wilmington, Delware) was added to purified water to make 2 wt % aqueous solution. The thermochromic black-colorless dye powder with thermal transition at 28° C. (Atlanta Chemical Engineering LLC, Tampa, FL), was added into pre-made 2 wt % polyvinylpyrolidine aqueous solution to make dye concentrations ranging from 0.6 wt % to 3 wt %. White SONTARA non-woven supplied by Glatfelter (Charlotte, NC) was soaked in the dye-polyvinylpyrolidine solutions for 5 minutes, then taken out and air dried. This SONTARA containing dye and adhesive laminate (3M Company, St Paul, MN) consisting of polyurethane film and acrylate adhesive were made into an experimental dressing prototype.

Freshly euthanized naïve young adult female mixed breed agricultural swine (Yorkshire X from Midwest Research Swine, Gibbon, MN) with minimal skin pigmentation and weighing 10-40 kg were used for this study. To minimize complications, hair and dirt on the swine skin at the intended application sites were removed prior to the study. A warming blanket (3M Company, St Paul, MN) was used to maintain the swine temperature throughout the study. A 20-gauge×1¼ ″Introcan Safety catheter (B. Braun, Bethlehem, PA) was inserted into the right abdominal area of the swine for subcutaneous injection and then the experimental dressing sample was applied to cover the insertion site. The dressing turned to SONTARA white color upon the dressing application, 0.9% sodium chloride injection solution (Patterson Veterinary, Loveland CO) was infused to the swine at a flow rate of 100 ml/hr subcutaneously to simulate infiltration. The dressing started to change to color gray-black at approximately 3-4 ml saline infusion near the tip of the catheter.

### Example 4

Thermochromic dye powder (Atlanta Chemical Engineering LLC, Tampa, FL) was added to an acrylic pressure-sensitive adhesive in ethyl acetate and heptane at ratio 1:1 (3M Company, St Paul, MN) at the concentrations shown in Table 1, mixed, and then coated on a primed 1 mil PET film (3M Company, St Paul, MN). The coated film was then dried at 70° C. for 10 minutes to obtain a dry pressure sensitive adhesive containing dye with thickness about 1 mil. The adhesive was then laminated to a PELLETHANE 5863-86A-VG film backing (Lubrizol, Wickliffe, OH) to provide adhesive film constructions having thermochromic dye in the adhesive.

Test specimens measuring 2.54 centimeters by 12.7 centimeters were cut from each adhesive film construction. The PET liner was removed from the adhesive and each test specimen was laminated to a test panel by placing the adhesive side of each specimen on a test panel and laminating using two passes of a 5-lbs roller in each direction. The test panel was #320 stainless steel. The peel test was carried out using a Zwick tensile tester (Z005) equipped with a 50 kg load cell at room temperature with a separation rate of 30.5 centimeters/minute. Each test was done in triplicate. The reported results are an average of 3 measurements reported as oz/inch and are summarized in Table 1.

TABLE 1
Weight percent of dyes in dry adhesive and results of peel
testing for test specimens made from the corresponding dye.

| | | | Adhesion |
|---|---|---|---|
| Sample | | Concentration | to steel |
| ID | Dye | dry (wt %) | (oz/inch) |
| EX-1 | Black to colorless at 28° C. | 1.60% | 11.2 ± 0.2 |
| EX-2 | Black to colorless at 28° C. | 3.20% | 12.0 ± 0.6 |
| EX-3 | Black to colorless at 28° C. | 6.40% | 11.3 ± 0.2 |
| EX-4 | Black to colorless at 28° C. | 9.60% | 11.6 ± 0.5 |
| EX-5 | Black to colorless at 31° C. | 1.60% | 12.2 ± 0.6 |
| EX-6 | Black to yellow at 31° C. | 1.60% | 12.2 ± 0.4 |
| EX-7 | Black to pink at 31° C. | 1.60% | 11.4 ± 0.3 |
| EX-8 | Red to colorless at 31° C. | 1.60% | 12.9 ± 0.7 |
| EX-9 | Red to yellow at 31° C. | 1.60% | 12.2 ± 0.6 |
| EX-10 | Blue to colorless at 31° C. | 1.60% | 11.8 ± 0.6 |
| EX-11 | Blue to colorless at 31° C. | 3.20% | 13.7 ± 1.2 |
| EX-12 | Blue to colorless at 31° C. | 6.40% | 12.8 ± 0.4 |
| EX-13 | Blue to colorless at 31° C. | 9.60% | 12.1 ± 1.1 |
| CE-1 | Control, no dye | NA | 13.9 ± 0.6 |

### Example 5

Thermochromic dye powder (Atlanta Chemical Engineering LLC, Tampa, FL) was added to water with polyglycerol, hydroxypropyl guar (Solvay, Princeton, NJ) and gamma irradiated polyvinyl pyrrolidone K90 (Ashland, Wilmington, DE). The mixture was then hot-pressed into a 30 mil thick gel sheet containing 0.85 wt % dye. The gels showed color changes with temperature, from dark blue to light blue or black to light gray.

### Example 6

Thermochromic paint solution containing a thermochromic dye having a black to colorless transition at 28° C. (Atlanta Chemical Engineering LLC, Tampa, FL) was Meyer rod (size 20) coated onto polyurethane film side (ESTANE 58237 TPU, Lubrizol, Wickliffe, OH) of an eight-inch-wide adhesive laminate consisting of polyurethane film, adhesive and release liner (3M Company, St Paul, MN), then dried in a 150° F. oven for two minutes. This black dye-coated adhesive laminate was then Meyer rod (size 20) coated with a second thermochromic paint solution and dried in a 150° F. oven for two minutes. The dye in the second thermochromic paint solution had a blue to colorless transition at 31° C. This two-layer dye-coated sample was cut 1 inch×1 inch pieces and then used to obtain color change vs. temperature curve.

A 1 inch×1 inch sample was placed on a hot plate increasing temperature from 25° C. to 40° C. Two videos were taken simultaneously, one recorded with a digital camera to capture the color change, and one with a thermal camera to capture the temperature change. For each video, a box selection was made for the background (plate corner) and the middle of each of the sample squares. Color values were then averaged within that selection. Each video pair was synchronized.

In order to gain a metric of perceived color change, the Euclidean distance of dressing color from the background is calculated in the CIELAB color space. The CIELAB color space was designed to be perceptibly uniform to the human eye, with various modifications and weights added over the years to handle various color applications and new corrections. A distance in this color space is a useful quantitative measure to determine the amount of dressing color change, how different a dressing color is from a constant background, and inform on how easily a dressing color change could be detected by an observer.

Color change was calculated with the following method. Both visible light and thermal imagery were recorded for warmed and cooled dressings. Areas of interest were chosen in the video frames that included the sample dressings and a section of a white background. Captured frames from the visible camera were converted to CIELAB color space with the OpenCV Python package, and averaged for each of the regions of interest. Once in CIELAB color space, the Euclidian distance was calculated between the background color and the sample dressing color and correlated to an associated temperature, taken from the IR camera video frames. The Euclidian distance between the color of the dressing and the color of the background is plotted on the y-axis and associated temperature on the x-axis. With this information, we can quantify the perceptible color change as the temperature of the dressing changes and compare between dressings of different transition temperatures and different color schemes.

The experimental dressing had two transition temperatures, one near 29° C., the other is near 33° C.

### Example 7: UV Cured Gel

A mixture of 8.1 g of poly(ethylene glycol) methyl ether acrylate with average molecular weight 480 (SIGMA-ALDRICH, St. Louis, MO), 5.9 g of 4-hydroxybutyl acrylate (BASF, Palmyra, MO), 4.0 grams of mixed dodecyl acrylate blend, 0.027 grams Irgacure 2959 photo-initiator, 0.009 grams of Irgacure 819 photo-initiator, and 0.18 g of thermochromic pigment (black to colorless transition at 28° C. powder (Atlanta Chemical Engineering LLC, Tampa, FL) were mixed on a oscillating table for at least 24 hours. The mixed dodecyl acrylate blend was prepared according to Example 9 of U.S. Pat. No. 9,102,774 (Clapper, et al.). The mixture was then coated and cured between two silicone coated polyester release liners to provide a cured adhesive film having thickness of approximately 0.25 mm. Curing was done using ultraviolet (UV) irradiation for approximately 30 minutes under an enclosed bank of six light bulbs of type 350 Blacklight F15 TB/350BL 15 W (SYLVANIA brand). The cured sample was mostly opaque and black in color at room temperature. The sample turned translucent white when heated in an oven at 60° C. for 10 minutes. The translucent sample had sufficient clarity that printed text was visible through the gel.

### Example 8

A mixture of 8.1 g of poly(ethylene glycol) methyl ether acrylate with average molecular weight 480 (SIGMA-ALDRICH, St. Louis, MO), 5.9 g of 4-hydroxybutyl acrylate (BASF, Palmyra, MO), 4.0 grams of mixed dodecyl acrylate blend, 0.027 grams Irgacure 2959 photo-initiator, and 0.009 grams of Irgacure 819 photo-initiator were mixed on a oscillating table for at least 24 hours and then coated and cured between two silicone coated polyester release liners. Curing was done using irradiation with ultraviolet (UV) rays for approximately 30 minutes under an enclosed bank of six light bulbs of type 350 Blacklight F15 TB/350BL 15 W (SYLVANIA brand).

Two layers of gel were prepared based on above procedure, one layer with thickness of 0.075 mm, another layer with thickness of 0.25 mm. The thick layer of gel was then patterned coated with thermochromic paint solution (black to colorless at 28° C., Atlanta Chemical Engineering LLC, Tampa, FL) by screening painting. The screen was a 50 micron thick polyester release liner that was 20% open with multiple 2.2 mm diameter holes. After coating, the sample was then dried in an oven at 60° C. for 60 minutes. The thin layer gel was then laminated to the screen-printed side of the thick gel sample to create a multilayer gel with a dried pigment layer between the two layers of gel. A polyurethane film of ESTANE 58237® TPU with thickness 0.9 mil (Lubrizol, Wickliffe, OH) was then laminated to the back side of the thicker gel layer. At room temperature, the sample was clear with black colored dots in it. Upon heating in an oven at 60° C. for 10 minutes, the dots of dried paint turned translucent white.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A medical dressing, comprising:
a backing layer comprising a first major surface and a second major surface opposite the first major surface;
a support material secured to the backing layer, wherein the support material is less elastic than the backing layer;
an adhesive on the second major surface of the backing layer;
a release liner; and
a thermochromic indicator having a color-change sensitivity at a temperature in a range between 20 degrees C. and 45 degrees C.

2. The medical dressing of claim 1, wherein the thermochromic indicator is selected from the group consisting of a dye, a colorant, an ink, and a pigment.

3. The medical dressing of claim 2, wherein the thermochromic indicator is a dye.

4. The medical dressing of claim 1, wherein the medical dressing is an IV site dressing.

5. (canceled)

6. The medical dressing of claim 1, further comprising a hydrogel island pad proximate the second major surface of the backing.

7. The medical dressing of claim 1, further comprising a carrier releasably attached to the first major surface of the backing layer.

8. The medical dressing of claim 1, further comprising an antimicrobial agent.

9. The medical dressing of claim 8, wherein the antimicrobial agent is selected from the group consisting of parachlorometaxylenol; triclosan; chlorhexidine and salts thereof; poly hexamethylene biguanide and salts thereof; iodine; idodophors; silver oxide; silver and its salts; octenidine; Olanexidine; peroxides; antibiotics; and combinations of the foregoing.

10. The medical dressing of claim 1, wherein the thermochromic indicator is incorporated in or located on at least one of the backing layer, the adhesive, the release liner, or the support material.

11. The medical dressing of claim 10, wherein the thermochromic indicator is transferred from the carrier to the backing layer upon removal of the carrier.

12. The medical dressing of claim 10, wherein the thermochromic indicator is printed on the medical dressing.

13. The medical dressing of claim 1, wherein the thermochromic indicator indicates a change in temperature from a first temperature indicative of a normal skin to a second temperature indicative of IV fluid infiltration.

14.-16. (canceled)

17. A medical dressing comprising:
a backing layer comprising a first major surface and a second major surface opposite the first major surface;
a carrier releasably attached to the first major surface of the backing layer;
an adhesive on the second major surface of the backing layer;
a release liner; and
a thermochromic indicator having a color-change sensitivity at a temperature in a range between 20 degrees C. and 45 degrees C.

18. The medical dressing of claim 17, wherein the thermochromic indicator is selected from the group consisting of a dye, a colorant, an ink, and a pigment.

19. The medical dressing of claim 17, wherein the medical dressing is an IV site dressing.

20. The medical dressing of claim 17, further comprising a hydrogel island pad proximate the second major surface of the backing.

21. The medical dressing of claim 17, further comprising an antimicrobial agent.

22. The medical dressing of claim 17, wherein the thermochromic indicator is transferred from the carrier to the backing layer upon removal of the carrier.

23. The medical dressing of claim 17, wherein the thermochromic indicator is printed on the medical dressing.

24. The medical dressing of claim 17, wherein the thermochromic indicator indicates a change in temperature from a first temperature indicative of a normal skin to a second temperature indicative of IV fluid infiltration.
